(19) United States
(12) Patent Application Publication
(10) Pub. No.: US 2012/0076811 A1
(43) Pub. Date: Mar. 29, 2012

(54) IMMUNODOMINANT COMPOSITIONS AND METHODS OF USE THEREFOR

(75) Inventor: Scheherazade Sadegh-Nasseri, Baltimore, MD (US)

(73) Assignee: The Johns hopkins University, Baltimore, MD (US)

(21) Appl. No.: 13/260,480

(22) PCT Filed: Mar. 26, 2010
(86) PCT No.: PCT/US10/28838

§ 371 Date: Dec. 7, 2011

Related U.S. Application Data
(60) Provisional application No. 61/163,657, filed on Mar. 26, 2009.

(51) Int. Cl. A61K 39/145; C07K 19/00; C12N 1/21; C12Q 1/70; C12N 15/44; C12N 15/63; C07K 7/08; A61P 31/16

(52) U.S. Cl. 424/186.1; 530/326; 530/327; 530/324; 435/5; 536/23.72; 435/320.1; 424/209.1; 435/252.33

(57) ABSTRACT

The present invention relates to immunodominant compositions, including immunodominant peptides of HA1 of influenza H5 hemagglutinin, polynucleotides encoding such peptides, and their methods of use. Such peptides are useful, for example, for the prevention, treatment and diagnosis of influenza.

### RELATED APPLICATION

The present patent application is the U.S. National Stage Application of International Application No. PCT/US10/028,838, filed on Mar. 26, 2010, which claims the benefit of U.S. Provisional Application Ser. No. 61/163,657, filed on Mar. 26, 2009; the entire contents of each of which application is incorporated herein by reference.

### GOVERNMENT INTEREST

This invention was made with Government support under grants awarded by NIAID (AI063764) and NIGM (GM053549). The Government has certain rights in the invention.

### BACKGROUND OF THE INVENTION

Typically between 250,000 and 500,000 people die each year as the result of influenza infection, with the death toll soaring into the millions in years in influenza pandemic years. Among the various strains of influenza, a particular avian strain, H5N1, has caused a great deal of concern due to its high lethality and its potential to produce a pandemic.

Although a wide variety of antibodies specific for many different influenza proteins are produced during an influenza infection, effective, protective immunity is primarily the consequence of high affinity antibodies to hemagglutinin (HA) and neuraminidase (NA). These high-affinity antibodies are usually the result antibody affinity maturation and heavy chain class switching in B cells. Efficient affinity maturation and heavy chain class switching is mediated by activated influenza-specific helper T cells. The ability to activate and/or detect influenza-specific helper T cells is therefore extremely useful in the prevention, treatment and detection of influenza.

Immunodominance is a phenomenon whereby a few specific peptides are selected as representative epitopes of a given protein antigen to the immune system, presumably for the purpose of easier immune regulation. These immunodominant peptides, when presented on Class II MHC by antigen presenting cells, are a significant contributor to helper T cell activation during viral infection, including influenza infection. Effort in identifying such epitopes has been ongoing because of their utility in the development of therapeutics against many diseases, including influenza.

### SUMMARY OF THE INVENTION

In some embodiments, the present invention relates to an isolated polypeptide of less than or equal to 35, 30, 25, 20 or 16 amino acids in length that includes an amino acid sequence that is at least 80%, 81%, 85%, 87%, 90%, 93%, 95% or 100% identical to a sequence selected from SEQ ID NOs: 1-29. In certain embodiments the polypeptide is covalently bonded to a carrier molecule, such as a non-influenza protein. In some embodiments the polypeptide is covalently and/or non-covalently bonded to a Class II MHC complex, including an HLA-DR complex such as HLA-DR1. In some embodiments the Class II MHC complex is multimeric (e.g., tetrameric), and/or is covalently bonded to a detectable label.

In certain embodiments, the invention relates to a pharmaceutical composition that includes one or more of the polypeptides of the invention. In some embodiments the polypeptide is covalently bonded to a carrier molecule, such as a non-influenza protein. In some embodiments the pharmaceutical composition also includes an adjuvant. Certain embodiments of the invention relate to a method of raising an immune response in a subject that includes administering a polypeptide of the invention and/or a pharmaceutical composition of the invention to the subject, including a subject that expresses a HLA-DR such as HLA-DR1.

Certain embodiments of the invention relate to a method of reducing the risk of influenza infection in a subject that includes administering a polypeptide of the invention and/or a pharmaceutical composition of the invention to the subject, including a subject that expresses a HLA-DR such as HLA-DR1.

Certain embodiments of the invention relate to a method of treating a subject infected by influenza that includes administering a polypeptide of the invention and/or a pharmaceutical composition of the invention to the subject, including a subject that expresses a HLA-DR such as HLA-DR1. In some embodiments the influenza has an H5 hemagglutinin. In certain embodiments the influenza is H5N1.

In some embodiments, the invention relates to a method of detecting exposure to influenza by a subject that includes the step of contacting CD4 T cells from the subject with polypeptide of the invention that is covalently bonded to a Class II MHC complex, such as a H5-DR complex, including H5-DR1. In some embodiments the influenza has an H5 hemagglutinin. In certain embodiments the influenza is H5N1. In certain embodiments the Class II MHC complex is multimeric, e.g., tetrameric, and/or is covalently linked to a detectable label. In some embodiments the method also includes the step of detecting proliferation of said CD4 T cells and/or detecting expression of a cytokine, such as IL-2 or IFN-γ, by said T cells. In certain embodiments the subject expresses a HLA-DR such as HLA-DR1.

Some embodiments of the invention relate to a kit that includes a polypeptide and/or a pharmaceutical composition of the invention. In certain aspects of the invention the kit also includes an adjuvant. In some embodiments, the polypeptide is covalently bonded to a carrier molecule.

Certain embodiments of the invention relate to an isolated nucleic acid encoding a polypeptide of the invention wherein said nucleic acid does not encode 36 consecutive amino acids of an HA1 of an influenza hemagglutinin, such as hemagglutinin H5.

Some embodiments of the invention relate to a vector that includes a nucleic acid of the invention, and/or a pharmaceutical composition containing a nucleic acid of the invention.

In some embodiments, the invention relates to a cell containing a nucleic acid of the invention. In certain embodiments the cell is a host cell, and/or an antigen presenting cell, such as a dendritic cell. In some embodiments the cell expresses an HLA-DR complex such as HLA-DR1. In certain embodiments the invention relates to a pharmaceutical composition that includes a cell of the invention.

### BRIEF DESCRIPTION OF THE DRAWINGS

FIG. 1 shows the results of an SDS-PAGE assay demonstrating little proteolysis of DR1 to cathepsin B and H under the conditions adopted for the digestion of HA1 recombinant protein.

FIG. 2 shows the results of an SDS-PAGE assay in which Empty DR1 molecules, pre-formed HA₃₀₆₋₃₁₈/DR1 complexes, and DM were incubated in the presence or absence of CatB and CatH (left panel) or CaS (right panel), demonstrating little proteolysis of DR1.

FIG. 3 shows the mass spectra of peptides eluted from DR1 after incubation with rHA1 in the presence of DM and cathepsins.

FIG. 4 shows the proliferation of lymphocytes harvested from rHA1 immunized DR1 transgenic mice after treatment with HA₃₀₆₋₃₁₈ peptide (A/Texas/1/77), HA₂₉₈₋₃₁₇ peptide (A/PR/8/34), human CLIP₈₉₋₁₀₅, or rHA1 protein.

FIG. 5 shows the proliferation of lymphocytes harvested from rHA1 immunized DR1 transgenic mice after treatment with HA₃₀₆₋₃₁₈ peptide (A/Texas/1/77), HA₂₉₈₋₃₁₇ peptide (A/PR/8/34), human CLIP₈₉₋₁₀₅, or rHA1 protein.

FIG. 6 shows the mass spectra of peptides eluted from DR1 after incubation with CII in the presence of DM and cathepsins.

FIG. 7 shows the mass spectra of FIG. 6 expanded between m/z 2800 and 3500 Da.

FIG. 8 shows the proliferation of lymphocytes harvested from CII immunized DR1 transgenic mice after treatment with CII₂₈₀₋₂₉₄, human CLIP₈₉₋₁₀₅, or CII protein.

FIG. 9 shows the mass spectra of peptides eluted from DR1 after incubation with H5N1-rHA1 in the presence of DM and cathepsins.

FIG. 10 shows CID fragmentation of peptides of FIG. 9 at m/z 1814.82 Da.

FIG. 11 shows CID fragmentation of peptides of FIG. 9 at m/z 2201.00 Da.

FIG. 12 shows the proliferation of lymphocytes harvested from H5N1-rHA1 immunized DR1 transgenic mice after treatment with HA₂₅₉₋₂₇₄, human CLIP₈₉₋₁₀₅, or H5N1-rHA1 protein.

FIG. 13 shows the IL-2 production at 24 or 48 hours of lymphocytes harvested from H5N1-rHA1 immunized DR1 transgenic mice after treatment with HA₂₅₉₋₂₇₄, human CLIP₈₉₋₁₀₅, or H5N1-rHA1 protein.

FIG. 14 shows the IFN-γ production at 48 or 72 hours of lymphocytes harvested from H5N1-rHA1 immunized transgenic mice after treatment with HA₂₅₉₋₂₇₄, human CLIP₈₉₋₁₀₅, or H5N1-rHA1 protein.

FIG. 15 shows FACS plots of lymphocytes harvested from DR1 transgenic mice that were either immunized with H5N1-rHA1 and CFA or CFA alone, where the cells were labeled with anti-CD44 antibody and either CLIP tetramer or H5N1 HA₂₅₉₋₂₇₄ tetramer.

FIG. 16 shows FACS plots of the cells from FIG. 15 that were stimulated with H5N1-rHA1 for an additional 7 days in vitro and labeled with anti-CD44 antibody and either CLIP tetramer or H5N1 HA₂₅₉₋₂₇₄ tetramer.

FIG. 17 shows the ribbon structure and amino acid sequence of H5N1-rHA1 (SEQ ID NO: 30).

### DETAILED DESCRIPTION OF THE INVENTION

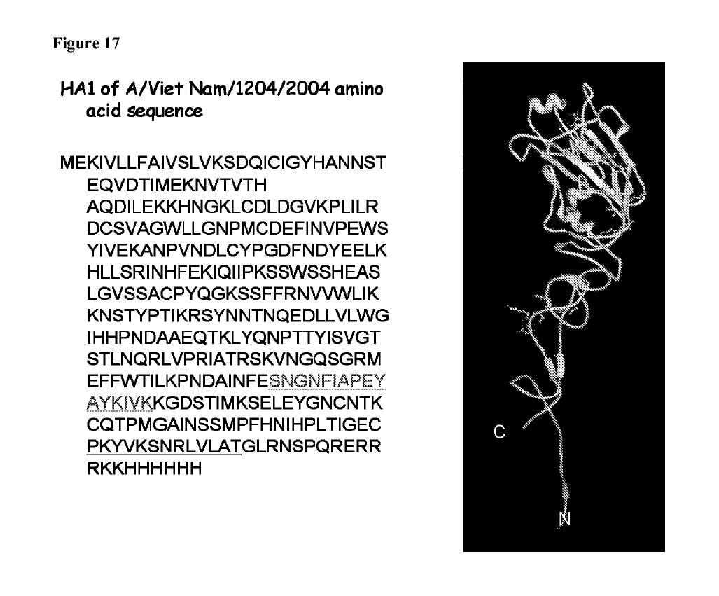

The present invention relates to, for example, immunodominant peptides from HA1 of influenza hemagglutinin H5 (FIG. 17). These immunodominant peptides were identified using a cell-free antigen processing system.

Such peptides, when presented on HLA-DR Class II MHC complexes are able to activate CD4⁺ helper T cells and thereby induce an immune response to influenza viruses, including influenza viruses that express hemagglutinin H5 such as H5N1. Such peptides are therefore useful, for example, as a vaccine for preventing or treating influenza infection (e.g., an H5N1 infection) or as a diagnostic for detecting exposure or infection by H5 hemagglutinin-expressing influenza, such as H5N1.

The immunodominant peptides of the present invention provide numerous advantages over conventional influenza vaccines, which typically use killed or attenuated whole virus. For example, because preparation of vaccines based on the peptides of the instant invention do not require the use of live virus, vaccines of the instant invention can be produced more rapidly and less expensively than conventional influenza vaccines. The absence of infectious material also renders the peptide-based vaccines of the instant invention safer than attenuated influenza based vaccines, particularly in immunocompramized individuals. Furthermore, because the immunodominant peptide based vaccines of the instant invention specifically target activation of helper T cells, such vaccines are able to efficiently induce B cells to produce the high affinity antibodies crucial for protection against influenza infection.

Because the phenomenon of immunodominance results in only a few specific peptides of a given protein antigen being presented to the immune system, vaccines based on non-immunodominant peptides are unable to efficiently induce a protective immune response. Immunodominant peptides, such as those provided in the instant invention, are therefore critical for the production of effective peptide based vaccines.

### DEFINITIONS

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of the foregoing.

The term “binding” or “interacting” refers to an association, which may be a stable association, between two molecules, e.g., between a polypeptide and a binding partner or agent, e.g., small molecule, due to, for example, electrostatic, hydrophobic, ionic and/or hydrogen-bond interactions under physiological conditions.

The term “HLA-DR” refers to a family of Class II MHC alleles. Included among this family of alleles is HLA-DR1. Non-limiting examples of other Class II HLA-DR alleles include HLA-DR1, HLA-DR3, HLA-DR4, HLA-DR7, HLA-DRB, HLA-DR9, HLA-DR10, HLA-DR11, HLA-DR12, HLA-DR13, HLA-DR14, HLA-DR15 and HLA-DR16 alleles.

A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid of the invention.

The term “isolated polypeptide” refers to a polypeptide, in certain embodiments prepared from recombinant DNA or RNA, or of synthetic origin, or some combination thereof, which (1) is not associated with proteins that it is normally found within nature, (2) is isolated from the cell in which it normally occurs, (3) is isolated free of other proteins from the same cellular source, (4) is expressed by a cell from a different species, or (5) does not occur in nature.

The term “isolated nucleic acid” refers to a polynucleotide of genomic, cDNA, or synthetic origin or some combination there of, which (1) is not associated with the cell in which the “isolated nucleic acid” is found in nature, or (2) is operably linked to a polynucleotide to which it is not linked in nature.

The terms “label” or “labeled” refer to incorporation or attachment, optionally covalently or non-covalently, of a detectable label into a molecule, such as a polypeptide.

A “patient”, “subject” or “host” refers to either a human or a non-human animal.

The term “percent identical” refers to sequence identity between two amino acid sequences or between two nucleotide sequences. Identity can each be determined by comparing a position in each sequence which may be aligned for purposes of comparison.

When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “polynucleotide” and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a non-natural arrangement.

The terms “polypeptide” and “peptide” are used interchangeably to refer to a polymeric form of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions may occur at the amino-terminus or carboxy-terminus of the reference polypeptide, or alternatively both. Fragments typically are at least 8 amino acids long, at least 16 amino acids long. A fragment can retain one or more of the biological activities of the reference polypeptide. In certain embodiments, a fragment may comprise core epitope of an immunodominant peptide that retrains the immunogenic properties of the immunodominant peptide.

The word “substantially” does not exclude “completely” e.g. a composition which is “substantially free” from Y may be completely free from Y. Where necessary, the word “substantially” may be omitted from the definition of the invention.

The term “substantially homologous,” when used in connection with amino acid sequences, refers to sequences which are substantially identical to or similar in sequence with each other, giving rise to a homology of conformation and thus to retention, to a useful degree, of one or more biological (including immunological) activities. The term is not intended to imply a common evolution of the sequences.

“Substantially purified” refers to a protein that has been separated from components which naturally accompany it. Preferably the protein is at least about 80%, more preferably at least about 90%, and most preferably at least about 99% of the total material (by volume, by wet or dry weight, or by mole percent or mole fraction) in a sample. Purity can be measured by any appropriate method, e.g., in the case of polypeptides, by column chromatography, gel electrophoresis or HPLC analysis.

### Immunodominant Peptides

Polypeptides of the invention include, but are not limited to, for example, polypeptides comprising the amino acid sequences listed in Table 1, including polypeptides comprising an amino acid sequence of SNGNFIAPEYAYKIVK (SEQ ID NO: 1) or SNGNFIAPEYAYKIVKKGDS (SEQ ID NO: 2). Polypeptides of the invention also include, but are not limited to, for example, polypeptides consisting of the amino acid sequences listed in Table 1, including polypeptides consisting of and consisting essentially of an amino acid sequence of SEQ ID NO: 1 or 2.

### Table 1

TABLE 1

| Polypeptide Sequence | SEQ ID NO. |
|---|---|
| SNGNFIAPEYAYKIVK | 1 |
| SNGNFIAPEYAYKIVKKGDS | 2 |
| SNGNFIAPEYAYKIV | 3 |
| SNGNFIAPEYAYKI | 4 |
| SNGNFIAPEYAYK | 5 |
| NGNFIAPEYAYKIVK | 6 |
| NGNFIAPEYAYKIV | 7 |
| NGNFIAPEYAYKI | 8 |
| GNFIAPEYAYKIVK | 9 |
| GNFIAPEYAYKIV | 10 |
| NFIAPEYAYKIVK | 11 |
| SNGNFIAPEYAYKIVKKGD | 12 |
| SNGNFIAPEYAYKIVKKG | 13 |
| SNGNFIAPEYAYKIVKK | 14 |
| NGNFIAPEYAYKIVKKGDS | 15 |
| GNFIAPEYAYKIVKKGDS | 16 |
| NFIAPEYAYKIVKKGDS | 17 |
| IAPEYAYKIVKKGDS | 18 |
| APEYAYKIVKKGDS | 19 |
| PEYAYKIVKKGDS | 20 |
| NGNFIAPEYAYKIVKKGD | 21 |
| NGNFIAPEYAYKIVKKG | 22 |
| NGNFIAPEYAYKIVKK | 23 |
| GNFIAPEYAYKIVKKGD | 24 |
| GNFIAPEYAYKIVKKG | 25 |
| GNFIAPEYAYKIVKK | 26 |
| NFIAPEYAYKIVKKGD | 27 |
| NFIAPEYAYKIVKKG | 28 |
| NFIAPEYAYKIVKK | 29 |

The polypeptides of the instant invention are efficiently presented on Class II MHC complexes, including HLA-DR complexes such as HLA-DR1. Because the peptide binding pocket of Class II MHC complexes are open at either end, additional peptides can be added to either end of the peptides listed in Table 1 without affecting its ability to be presented on Class II MHC or activate helper T cells. For example, 1, 2, 3, 4, 5, 6, 7, 8, 9, 10, 11, 12, 13, 14 or 15 amino acids can be added to either the N terminus or the C terminus of the peptides listed in Table 1 without affecting their ability to be presented on Class II MHC and act as immunodominant peptides. The additional amino acids may be identical to the corresponding amino acids of the influenza H5N1 HA1 protein of FIG. 17. However, the additional amino acids have a minimal contribution to the interaction between a T cell receptor and the peptide/MHC complex, and therefore can be different from the corresponding amino acids of the influenza H5N1 HA1 protein without affecting its ability to be presented on Class II MHC or activate helper T cells.

Polypeptides of the invention may comprise amino acid sequences that have sequence identity to the amino acid sequences listed in Table 1. Depending on the particular sequence, the degree of sequence identity is preferably greater than or equal to 50%, 60%, 70%, 75%, 80%, 81%, 82%, 83%, 84% 85%, 86%, 87%, 88%, 89%, 90%, 91%, 92%, 93%, 94%, 95%, 96% or 100%. Identity between polypeptides is preferably determined by the Smith-Waterman homology search algorithm as implemented in the MPSRCH program (Oxford Molecular), using an affine gap search with parameters gap open penalty=12 and gap extension penalty=1.

These polypeptides may, compared to the sequences of Table 1, include one or more (e.g. 1, 2, 3, 4, 5, 6, 7, 8, 9, 10, 11, 12, 13, 14 or 15) conservative amino acid substitutions i.e. replacements of one amino acid with another which has a related side chain. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. Moreover, the polypeptides may have one or more (e.g. 1, 2, 3, 4, 5 or 6) single amino acid deletions relative to a reference sequence. Furthermore, the polypeptides may include one or more (e.g. 1, 2, 3, 4, 5 or 6) insertions (e.g. each of 1, 2 or 3 amino acids) relative to a reference sequence.

Peptides as short as 8 amino acids in length can form a complex with Class II MHC and activate helper T cells. Therefore, certain embodiments of the invention include peptide fragments as short as 8, 9 or 10 amino acids of the sequences listed in Table 1. Such fragments can be the result of a deletion of either C terminal amino acids, N terminal amino acids, or both N and C terminal amino acids. Such fragments may comprise at least n consecutive amino acids from the sequences and, depending on the particular sequence, n is 5 or more (e.g. 8, 9, 10, 11, 12, 13, 14, 15, 16, 17 or 18).

Polypeptides of the invention comprising the sequences listed in Table 1 may be less than or equal to 15, 16, 17, 18, 19, 20, 21, 22, 23, 24, 25, 26, 27, 28, 29, 30, 31, 32, 33, 34 or 35 amino acids long.

Immunodominant polypeptides of the invention can be prepared using any method known in the art. For example, the peptides of the instant invention can be made by chemical synthesis (in whole or in part), by digesting longer polypeptides using proteases, by translation from RNA, by purification from cell culture (e.g. from recombinant expression), etc. For example, peptides can be produced by in vitro chemical synthesis, such as solid-phase peptide synthesis (e.g. methods based on tBoc or Fmoc chemistry). Enzymatic synthesis may also be used in part or in full. As an alternative to chemical synthesis, biological synthesis may be used e.g. the polypeptides may be produced by translation. This may be carried out in vitro or in vivo. Biological methods are in general restricted to the production of polypeptides based on L-amino acids, but manipulation of translation machinery (e.g. of aminoacyl tRNA molecules) can be used to allow the introduction of D-amino acids (or of other non natural amino acids, such as iodotyrosine or methylphenylalanine, azidohomoalanine, etc.). Polypeptides of the invention may have covalent modifications at the C-terminus and/or N-terminus.

Polypeptides of the invention can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.).

Polypeptides of the invention may be provided in purified or substantially purified form i.e. substantially free from other polypeptides (e.g. free from naturally-occurring polypeptides), and may be at least about 50%, 60%, 70%, 80%, 90%, 91%, 92%, 93%, 94%, 95%, 96%, 97%, 98% or 99% pure (by weight).

Polypeptides of the invention may be attached to a solid support. Polypeptides of the invention may comprise a detectable label (e.g. a radioactive or fluorescent label, or a biotin label).

Polypeptides of the present invention can be either covalently or non-covalently bonded to a Class II MHC complex, including a HLA-DR complex such as HLA-DR1. Such complexes may be in monomeric form or in multimeric form, such as tetrameric form. The peptide/MHC complexes of the instant invention may be labeled with a detectable marker, or may be attached to a solid support.

The polypeptides of the present invention may be covalently bonded to heterologous proteins and/or carrier molecules.

The invention provides a process for producing polypeptides of the invention, comprising the step of culturing a host cell of the invention under conditions which induce polypeptide expression.

The invention provides a process for producing a polypeptide of the invention, wherein the polypeptide is synthesized in part or in whole using chemical means.

### Nucleic Acids Encoding Immunodominant Peptides

The invention also includes nucleic acids encoding the polypeptides of the invention. The nucleic acid of the invention may comprise nucleotide sequences that encode polypeptides of the present invention, including polypeptides comprising a sequence listed in Table 1. In certain embodiments, said nucleic acid does not encode 40, 39, 38, 37, 36, 35, 34, 33, 32, 31, 30, 19, 18, 17 or 16 consecutive amino acids of an influenza hemagglutinin, such as influenza hemagglutinin H5, or HA1 of influenza hemagglutinin H5.

The invention also provides nucleic acids comprising nucleotide sequences having sequence identity to such nucleotide sequences. Identity between sequences may be determined by the Smith-Waterman homology search algorithm. Depending on the particular sequence, the degree of sequence identity is preferably greater than 50% (e.g. 60%, 70%, 75%, 80%, 85%, 90%, 91%, 92%, 93%, 94%, 95%, 96%, 97%, 98%, 99% or more). The invention includes nucleic acid comprising sequences complementary to these sequences (e.g. for antisense or probing, or for use as primers).

Nucleic acids according to the invention can take various forms (e.g. single-stranded, double-stranded, vectors, primers, probes, labeled etc.).

Nucleic acids of the invention may be provided in purified or substantially purified form i.e. substantially free from other nucleic acids (e.g. free from naturally-occurring nucleic acids), generally being at least about 50%, 60%, 70%, 80%, 90%, 91%, 92%, 93%, 94%, 95%, 96%, 97%, 98% or 99% pure (by weight). Nucleic acids of the invention can be prepared in many ways e.g. by chemical synthesis (at least in part), by digesting longer nucleic acids using nucleases (e.g. restriction enzymes), by joining shorter nucleic acids (e.g. using ligases or polymerases), from genomic or cDNA libraries, etc.

Nucleic acids of the invention may be part of a vector i.e. part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector.

In certain embodiments, the present invention includes cells comprising nucleic acids of the invention. Such cells can be, for example, host cells or antigen presenting cells such as dendritic cells, macrophages or B cells. In some embodiments the cells of the invention express HLA-DR complexes, such as HLA-DR1. Such cells are useful, for example, for the production of the peptides of the invention or as a whole cell vaccine. The cells of the instant invention can therefore be a component in a pharmaceutical composition.

### Pharmaceutical Compositions

The invention provides compositions comprising: (a) polypeptide or nucleic acid of the invention; and (b) a pharmaceutically acceptable carrier. These compositions may be suitable, for example, as immunogenic compositions, as diagnostic reagents, or as vaccines. Vaccines according to the invention may either be prophylactic (i.e., to prevent infection) or therapeutic (i.e., to treat infection).

The polypeptide or nucleic acid of the invention is an active ingredient in the composition, and may be present at a therapeutically effective amount i.e. an amount sufficient to reduce the likelihood of, prevent or treat an influenza infection. The precise effective amount for a given patient will depend upon their size and health, the nature and extent of infection, and the composition or combination of compositions selected for administration. The effective amount can be determined by routine experimentation and is within the judgment of the clinician. Polypeptides may be included in the composition in the form of salts and/or esters.

The pharmaceutical compositions of the invention may include multiple immunodominant polypeptides of the instant invention. In certain embodiments, the pharmaceutical compositions of the invention include additional influenza peptides or whole attenuated or killed influenza viruses. As such, the pharmaceutical compositions of the instant invention are useful when administered in conjunction with additional molecules that induce immunity to influenza, such as other influenza vaccines.

A “pharmaceutically acceptable carrier” includes any carrier that does not itself induce the production of antibodies harmful to the individual receiving the composition. Suitable carriers may be large, slowly metabolized macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier.

Compositions of the invention may comprise detergent e.g. a Tween (polysorbate), such as Tween 80. Detergents are generally present at low levels e.g. <0.01%.

Compositions of the invention may include sodium salts (e.g. sodium chloride) to give tonicity.

Compositions of the invention may include a buffer, for example, a phosphate buffer.

Compositions of the invention may comprise a sugar alcohol (e.g. mannitol) or a disaccharide (e.g. sucrose or trehalose) e.g. at around 15-30 mg/ml (e.g. 25 mg/ml), particularly if they are to be lyophilized or if they include material which has been reconstituted from lyophilized material. The pH of a composition for lyophilisation may be adjusted to around 6.1 prior to lyophilisation.

Polypeptides of the invention may be administered in conjunction with other immunoregulatory agents. In particular, compositions may include a vaccine adjuvant. Adjuvants which may be used in compositions of the invention include, but are not limited to, mineral-containing compositions, oil emulsions, saponin formulations, virosomes and virus-like particles, bacterial or microbial derivatives, immunostimulatory oligonucleotides, human immunomodulators bioadhesives and mucoadhesives, microparticles, polyoxyethylene ether and polyoxyethylene ester formulations, liposomes, polyphosphazene (PCPP), muramyl peptides, imidazoquinolone compounds, thiosemicarbazone compounds, and tryptanthrin compounds.

Mineral containing compositions suitable for use as adjuvants in the invention include mineral salts, such as aluminium salts and calcium salts. The invention includes mineral salts such as hydroxides (e.g. oxyhydroxides), phosphates (e.g. hydroxyphosphates, orthophosphates), sulphates, or mixtures of different mineral compounds (e.g. a mixture of a phosphate and a hydroxide adjuvant, optionally with an excess of the phosphate), with the compounds taking any suitable form (e.g. gel, crystalline, amorphous), and with adsorption to the salt(s) being preferred. Mineral containing compositions may also be formulated as a particle of metal salt.

Oil emulsion compositions suitable for use as adjuvants in the invention include squalene-water emulsions, such as MF59 (5% Squalene, 0.5% Tween 80, and 0.5% Span 85, formulated into submicron particles using a microfluidizer). MF59 is used as the adjuvant in the FLUAD™ influenza virus trivalent subunit vaccine.

Oil emulsion compositions suitable for use as adjuvants in the invention also include submicron oil-in-water emulsions. Preferred submicron oil-in-water emulsions for use herein are squalene/water emulsions, optionally containing varying amounts of MTP-PE, such as a submicron oil-in-water emulsion containing 4-5% w/v squalene, 0.25-1.0% w/v Tween 80 (polyoxyethylenesorbitan monooleate), and/or 0.25-1.0% Span 85 (sorbitan trioleate), and, optionally, N-acetylmuramyl-L-alanyl-D-isoglutaminyl-L-alanine-2-(1′-2′-dipalmitoyl-s-n-glycero-3-hydroxyphosphosphoryloxy)-ethylamine (MTP-PE).

Saponin formulations may also be used as adjuvants in the invention. Saponins are a heterologous group of sterol glycosides and triterpenoid glycosides that are found in the bark, leaves, stems, roots and even flowers of a wide range of plant species. Saponins isolated from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs.

Saponin compositions have been purified using HPLC and RP-HPLC. Specific purified fractions using these techniques have been identified, including QS7, QS17, QS18, QS21, QH-A, QH-B and QH-C. Saponin formulations may also comprise a sterol, such as cholesterol.

Combinations of saponins and cholesterols can be used to form unique particles called immunostimulating complexes (ISCOMs). ISCOMs typically also include a phospholipid such as phosphatidylethanolamine or phosphatidylcholine. Any known saponin can be used in ISCOMs. Preferably, the ISCOM includes one or more of QuilA, QHA and QHC. Optionally, the ISCOMS may be devoid of additional detergent(s).

Virosomes and virus-like particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1).

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof. Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. Lipid A derivatives include derivatives of lipid A from Escherichia coli such as OM-174.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory. The CpG's can include nucleotide modifications/analogs such as phosphorothioate modifications and can be double-stranded or single-stranded.

The CpG sequence may be directed to TLR9, such as the motif GTCGTT or TTCGTT. The CpG sequence may be specific for inducing a Th1 immune response, such as a CpG-A ODN, or it may be more specific for inducing a B cell response, such a CpG-B ODN.

The CpG oligonucleotide may be constructed so that the 5′ end is accessible for receptor recognition. Optionally, two CpG oligonucleotide sequences may be attached at their 3′ ends to form “immunomers.”

Human immunomodulators suitable for use as adjuvants in the invention include cytokines, such as interleukins (e.g. IL-1, IL-2, IL-4, IL-5, IL-6, IL-7, IL-12, etc.), interferons (e.g. interferon-gamma), macrophage colony stimulating factor, and tumor necrosis factor.

Bioadhesives and mucoadhesives may also be used as adjuvants in the invention. Suitable bioadhesives include esterified hyaluronic acid microspheres or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention.

Microparticles may also be used as adjuvants in the invention. Microparticles (i.e. a particle of about 100 nm to about 150 μm in diameter, formed from materials that are biodegradable and non-toxic (e.g. a poly(α-hydroxy acid), a polyhydroxybutyric acid, a polyorthoester, a polyanhydride, a polycaprolactone), with poly(lactide-co-glycolide) may be used, optionally treated to have a negatively-charged surface (e.g. with SDS) or a positively-charged surface (e.g. with a cationic detergent, such as CTAB).

Adjuvants suitable for use in the invention include polyoxyethylene ethers and polyoxyethylene esters. Such formulations further include polyoxyethylene sorbitan ester surfactants in combination with an octoxynol as well as polyoxyethylene alkyl ethers or ester surfactants in combination with at least one additional non-ionic surfactant such as an octoxynol. Polyoxyethylene ethers may be, for example, selected from the following group: polyoxyethylene-9-lauryl ether (laureth 9), polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether.

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-m-glycero-3-hydroxyphosphoryloxy)-ethylamine MTP-PE).

Examples of imidazoquinolone compounds suitable for use adjuvants in the invention include Imiquamod and its homologues (e.g. “Resiquimod 3M”).

The invention may also comprise combinations of one or more of the adjuvants identified above. For example, the following combinations may be used as adjuvant compositions in the invention: (1) a saponin and an oil-in-water emulsion; (2) a saponin (e.g. QS21)+a non-toxic LPS derivative (e.g. 3dMPL); (3) a saponin (e.g. QS21)+a non-toxic LPS derivative (e.g. 3dMPL)+a cholesterol; (4) a saponin (e.g. QS21)+3dMPL+IL-12 (optionally +a sterol); (5) combinations of 3dMPL with, for example, QS21 and/or oil-in-water emulsions; (6) SAF, containing 10% squalene, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), (e.g., MPL+CWS (Detox™)); (8) one or more mineral salts (such as an aluminum salt)+a non-toxic derivative of LPS (such as 3dMPL); and (9) one or more mineral salts (such as an aluminum salt)+an immunostimulatory oligonucleotide (such as a nucleotide sequence including a CpG motif).

Compositions may be presented in vials, or they may be presented in ready-filled syringes. The syringes may be supplied with or without needles. A syringe will include a single dose of the composition, whereas a vial may include a single dose or multiple doses. Injectable compositions will usually be liquid solutions or suspensions. Alternatively, they may be presented in solid form (e.g. freeze-dried) for solution or suspension in liquid vehicles prior to injection.

Compositions of the invention may be packaged in unit dose form or in multiple dose form. For multiple dose forms, vials are preferred to pre-filled syringes. Effective dosage volumes can be routinely established.

Where a composition of the invention is to be prepared extemporaneously prior to use (e.g. where a component is presented in lyophilized form) and is presented as a kit, the kit may comprise two vials, or it may comprise one ready-filled syringe and one vial, with the contents of the syringe being used to reactivate the contents of the vial prior to injection.

Immunogenic compositions used as vaccines comprise an immunologically effective amount of a peptide of the invention, as well as any other components, as needed. By “immunologically effective amount”, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesize antibodies, the degree of protection desired, the formulation of the vaccine, the treating doctor's assessment of the medical situation, and other relevant factors.

Compositions of the invention may be administered directly to a patient. Direct delivery may be accomplished by parenteral injection (e.g. subcutaneously, intraperitoneally, intravenously, intramuscularly, or to the interstitial space of a tissue), or by rectal, oral, vaginal, topical, transdermal, intranasal, sublingual, ocular, aural, pulmonary or other mucosal administration. Intramuscular administration to the thigh or the upper arm is preferred. Injection may be via a needle (e.g. a hypodermic needle), but needle-free injection may alternatively be used.

Dosage treatment can be a single dose schedule or a multiple dose schedule. Multiple doses may be used in a primary immunization schedule and/or in a booster immunization schedule. A primary dose schedule may be followed by a booster dose schedule. A nucleic acid of the present invention may be used as a primer followed by a polypeptide of the present invention as a booster. Suitable timing between priming doses (e.g. between 4-16 weeks), and between priming and boosting, can be routinely determined.

### Methods of Using Immunodominant Peptides

The immunodominant peptides of the present invention are useful in methods of preventing or treating influenza and useful in diagnostic assays for detecting exposure to influenza in a mammal, including humans. The immunodominant peptides of the invention are presented to T cells on HLA-DR Class II MHC molecules, including HLA-DR1. The immunodominant peptides of the invention are therefore useful in methods of preventing or treating influenza and useful in diagnostic assays for detecting exposure to influenza in subjects that express HLA-DR, including in subjects that express HLA-DR1.

It can be determined whether a subject expresses HLA-DR including HLA-DR1 using routine methods that are well known in the art. For example, commercially available antibodies specific to particular HLA-DR alleles can be used to detect the presence of a particular HLA-DR allele, including HLA-DR1, in a blood sample taken from a subject. The presence of a particular HLA-DR allele can also be detected using PCR, DNA sequencing, on any other method known in the art.

In a diagnostic assay, the immunodominant peptides of the invention are useful in the detection of helper immune response against influenza viruses that express hemagglutinin H5, including H5N1. This immune response may be used, for example, as an indicator of exposure to influenza viruses that express hemagglutinin H5, including H5N1. This immune response can also be used to determine the strain of influenza that is infecting a subject. As the development of helper T cell responses may be an earlier event than the development of detectable antibodies against the protein, detection of helper T cell responses against the immunodominant peptides of the invention are useful in early influenza exposure detection.

In one embodiment, the immunodominant peptides of the invention are complexed with Class II MHC complexes, such as HLA-DR, and applied to a substrate or solid support. In certain embodiments the immunodominant peptides are covalently linked to the Class II MHC molecule. Lymphocytes from a subject are grown in the presence of the peptides in parallel with a control peptide such as a peptide. Specific cytokine release is then measured using such techniques such as ELISPOT and ELISA, or the lymphocytes are tested for helper T cell proliferation. Detection of an enhanced helper T cell immune response in comparison to the negative controls is indicative of exposure to influenza by the subject.

In another embodiment, the immunodominant peptides of the invention are complexed with Class II MHC complexes, such as HLA-DR. In certain embodiments the immunodominant peptides are covalently linked to the Class II MHC molecule. In certain other embodiments, the peptide/MHC complex may be labeled with a detectable marker. In some embodiments, the peptide/MHC complex is in the form of a multimer, such as a tetramer. This peptide/MHC complex may be used to detect the presence of influenza-specific helper T cells in a sample from a subject using, for example, flow cytometry of fluorescent microscopy.

The immunodominant peptide is also useful as a vaccine for inducing influenza-specific immunity against influenza virus, including influenza virus expressing hemagglutinin H5, such as H5N1. Such vaccines can be used in both a subject that is not infected by influenza in order to prevent future infection or in a subject that is infected by influenza in order to treat the influenza infection. The vaccines of the invention are also useful in preventing and/or treating influenza infections in individuals who have been exposed to influenza, such as H5N1, but that do not yet know their infection status.

In certain embodiments the present invention provides a method of enhancing the influenza-specific immunity of a subject comprising administering at least one immunodominant peptide of the invention or a vaccine composition comprising said peptide, variant, or derivative for a time and under conditions sufficient to induce an immune response in the subject. In certain embodiments, the peptide or vaccine composition is administered for a time and under conditions sufficient to elicit or enhance the expansion of CD4⁺ T cells.

In some embodiments, the peptide or vaccine composition is administered for a time and under conditions sufficient for influenza (e.g. H5N1)-specific cell mediated immunity to be enhanced in the subject.

The effective amount of peptide to be administered, either solus or in a vaccine composition varies with the route of administration, the weight, age, sex, or general health of the subject immunized, and the nature of the CTL response sought. All such variables are empirically determined by art-recognized means.

The peptide, optionally formulated with any suitable or desired carrier, adjuvant, BRM, or pharmaceutically acceptable excipient, is conveniently administered in the form of an injectable composition. Injection may be intranasal, intramuscular, sub-cutaneous, intravenous, intradermal, intraperitoneal, or by other known route. For intravenous injection, it is desirable to include one or more fluid and nutrient replenishers.

The optimum dose to be administered and the preferred route for administration may be established using animal models, such as, for example, by injecting a mouse, rat, rabbit, guinea pig, dog, horse, cow, goat or pig, with a formulation comprising the peptide, and then monitoring the immune response to the peptide and/or influenza resistance using any conventional assay.

Certain aspects of the invention include a method of providing or enhancing immunity against influenza, including H5N1, in an uninfected human subject comprising administering to said subject an immunologically active peptide comprising an immunodominant peptide of the invention or a vaccine composition comprising said peptide under conditions sufficient to provide immunological memory against a future infection by influenza, including H5N1.

Accordingly, this aspect of the invention provides for the administration of a prophylactic vaccine to the subject, wherein the active substituent of said vaccine (i.e. the epitope or polyepitope of the invention) induces immunological memory via memory T cells in an uninfected individual.

### Methods of Using Nucleic Acids Encoding Immunodominant Peptides

Nucleic acids of the invention can be used, for example, to produce polypeptides; as hybridization probes for the detection of nucleic acid in biological samples; to generate additional copies of the nucleic acids; to generate ribozymes or antisense oligonucleotides; as single-stranded DNA primers or probes; or as triple-strand forming oligonucleotides.

The nucleic acids of the invention may also be used for nucleic acid immunization. Nucleic acid immunization is now a developed field that has been applied to, for example, Neisseria meningitidis vaccines.

During nucleic acid immunization, the nucleic acid encoding the polypeptide of the invention may be expressed in vivo after delivery to a patient, at which point the expressed polypeptide then stimulates the immune system. The active ingredient will often take the form of a nucleic acid vector comprising: (i) a promoter (e.g. a CMV promoter); (ii) a sequence encoding the polypeptide, operably linked to the promoter; and optionally (iii) a selectable marker. Vectors may further comprise (iv) an origin of replication; and (v) a transcription terminator downstream of and operably linked to (ii). In general, (i) & (v) will be eukaryotic and (iii) & (iv) will be prokaryotic.

Vectors useful in nucleic acid immunization may include a prokaryotic marker for selection of the vector, a prokaryotic origin of replication, but a eukaryotic promoter for driving transcription of the polypeptide-encoding sequence. Such vectors will therefore (a) be amplified and selected in prokaryotic hosts without polypeptide expression, but (b) be expressed in eukaryotic hosts without being amplified. This arrangement is ideal for nucleic acid immunization vectors.

Vectors useful in nucleic acid immunization may include unmethylated CpG motifs e.g. unmethylated DNA sequences which have in common a cytosine preceding a guanosine, flanked by two 5′ purines and two 3′ pyrimidines. In their unmethylated form these DNA motifs have been demonstrated to be potent stimulators of several types of immune cell.

Such vectors can be delivered using gene delivery vehicles. The gene delivery vehicle can be of viral or non-viral origin.

Viral-based vectors for delivery of a desired nucleic acid and expression in a desired cell are well known in the art. Exemplary viral-based vehicles include, but are not limited to, recombinant retroviruses, alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532); hybrids or chimeras of these viruses may also be used), poxvirus vectors (e.g. vaccinia, fowlpox, canarypox, modified vaccinia Ankara, etc.), adenovirus vectors, and adeno-associated virus (AAV) vectors. Administration of DNA linked to killed adenovirus can also be employed.

Non-viral delivery vehicles and methods can also be employed, including, but not limited to, polycationic condensed DNA linked or unlinked to killed adenovirus alone ligand-linked DNA, eukaryotic cell delivery vehicles cells and nucleic charge neutralization or fusion with cell membranes. Naked DNA can also be employed. Liposomes (e.g. immunoliposomes) can also be used as gene delivery vehicles. Delivery DNA using PLG {poly(lactide-co-glycolide)} microparticles may be used e.g. by adsorption to the microparticles, which are optionally treated to have a negatively-charged surface (e.g. treated with SDS) or a positively-charged surface (e.g. treated with a cationic detergent, such as CTAB).

### Exemplification

### Materials and Methods

### Peptides

The influenza virus HA₃₀₆₋₃₁₈ peptide (PKYVKQNTLKLAT), its single amino acid substitution variant, HA_{Y308A} (PKAVKQNTLKLAT), (bold and underlining indicate substitution of the anchor residue from Tyrosine to Alanine), human CLIP₈₉₋₁₀₅ peptide (KMRMATPLLMQALPM), and CII₂₈₀₋₂₉₄ (AGFKGEQGPKGEPGP) were synthesized by Global peptide (Fort Collins, Colo.). HA₂₉₈₋₃₁₇ peptide (INSSLPYQNIHPVTIGECPKY), and HA₂₅₉₋₂₇₄ peptide (SNGNFIAPEYAYKIVK) were synthesized by Elim Biopharmaceuticals (Hayward, Calif.) at >85% purity as analyzed by reversed phase HPLC, and their identities were confirmed by mass spectrometry.

### Production of Recombinant Proteins

Soluble HLA-DR1*0101 was produced in baculovirus-transduced insect cells as described in Stern and Wiley, Cell 68:465-477 (1992). Soluble HLA-DR was expressed in the same manner and affinity-purified with M2 mAb sepharose (Sigma) at pH 6.0 through the FLAG tag placed on the C-terminus of the α chain.

Recombinant influenza hemagglutinin (rHA1) was produced in E. coli transformed with an expression vector for a dual 6× histidine-tagged influenza hemagglutinin. The protein contains residues 12˜340 of the hemagglutinin of Influenza strain A/PR/8/34 HA gene with a MRGSHHHHHHTDPSSRSA tag on the N-terminus and a ACPKYVKQNTLKLATGMRKLHHHHHHN tag on the C-terminus (the underlined residues comprise the HA₃₀₆₋₃₁₈ epitope from Influenza strain A/Texas/1/77). Following affinity purification from bacterial lysate with Ni-NTA-charged agarose resin (Ni-NTA Superflow, Qiagen) under denaturing and reducing conditions according to manufacturer protocols, the protein was refolded by stepwise dialysis into PBS/10% sucrose and then stored at −80° C. H5N1-rHA1 (amino acid 1-345) from strain A/Vietnam/1203/2005 was purified from 293 cells (eEnzyme).

### Fragmentation of Bovine Type II Collagen by Matrix Metalloproteinase 9

Bovine type II collagen (bCII, Chondrex) was heat-denatured and digested with Matrix Metalloproteinase 9 (MMP9; Calbiochem) by 4 hr incubation at 37° C. in MMP9 reaction buffer (100 mM Tris-HCl pH 7.5, 100 mM NaCl, 10 mM CaCl₂, 0.01% Tween 20 and 0.05% sodium azide). MMP9 was activated by incubating with Matrix Metalloproteinase 3 (MMP3, Sigma) for 22 hrs at 37° C. in MMP9 reaction buffer at a 100 MMP9:1 MMP3 molar ratio. MMP3 was activated by incubating overnight at 37° C. with 1.5 mM 4-aminophenylmercuric acetate (APMA; Sigma-Aldrich) in MMP3 storage buffer (50 mM Tris-HCl pH 7.6, 150 mM NaCl, 10 mM CaCl₂, 0.05% Brij 35, 0.02% sodium azide).

### Mass Spectrometry

### Sample Preparation:

HLA-DR1 (DR1), antigen, and HLA-DR (DM) were incubated in citrate phosphate buffer (pH 5.0) at 37° C. for ˜3 h, after which cathepsin B (bovine spleen, Sigma) and cathepsin H (human liver, Calbiochem) or cathepsin B, cathepsin H, and cathepsin S (human, Calbiochem) were added with 6 mM L-Cysteine and 4 mM EDTA for an additional 2-3 h. After this, the pH was adjusted to 7.5, 10 mM iodoacetamide was added, and DR1 was immunoprecipitated with Sepharose conjugated with DR1 specific mAb (L243). Bound peptides were eluted with TFA, filtered through a 10 kDa MWCO Microcon (Millipore), and lyophilized. For type II collagen data, eluted peptides were analyzed on the Axima-CFR MALDI-TOF mass spectrometer (Kratos Analytical, Shimadzu) with data acquired in reflectron mode. The matrix used was α-Cyano-4-hydroxycinnamic acid. Data were analyzed with LAUNCHPAD™ (Shimadzu/Kratos Analytical) and FindPept (See website Expasy website, http://us.expasy.org/tools/findpept.html).

vMALDI-LTQ Analysis.

Lyophilized samples were re-suspended in 5-10 ul 50% ethanol/50% water/0.2% TFA. 0.5 ul of re-suspended sample was spotted, dried, and covered with 0.5 uL matrix (40-50 mg/ml 2,5-dihydroxybenzoic acid or 2.5 mg/ml α-cyano-4-hydroxycinnamic acid in 50% ethanol/50% water/0.15% TFA). Samples were run on the vMALDI-LTQ mass spectrometer (ThermoFisher, San Jose, Calif.) with 400-micron fiber, using Tune Plus 2.2 Xcalibur 2.0 SR2 vMALDI LTQ 2.2 with Microsoft Windows XP service pack 2 software. Full MS scans (1100-4000 Da m/z) were acquired to select peptides of interest for identification by CID fragmentation (MS²). A subset of fragmented ions was selected for CID fragmentation (MS³) to confirm peptide sequence assignments. Spectra were manually acquired using CPS plate motion, 2 microscans, 0 sweep scans, automatic gain control (AGC) feature “on”, automatic scan filtering (ASF) “on” and set on 3000 for Full MS and 200 for MSⁿ, and accumulating 10-15 scans for Full MS and 15-25 scans for MS2 or MS3 spectra.

Data analysis included a) visual analysis of Full MS spectra to find the peptides of interest, and b) MSⁿ data collected for those peptides searched with Bioworks 3.3.1 SP1 (ThermoFisher) against a custom-built database containing all protein components present in the sample, with no enzyme, monoisotopic precursor and fragment ions, with tolerance for 2 missed cleavages, mass tolerance of 2 Da for MS¹, mass tolerance of 1 Da for MS² and MS³, and with allowances for variable amino acid modifications (carboamidomethylated cysteine and oxidation of methionine results in methionine with 48 Da loss for MS³ data).

### LTQ Analysis:

Lyophilized samples were re-suspended in water/1% TFA/0.2% acetonitrile, and 5 ul of re-suspended sample were loaded on nano-LC-ESI/MS/MS LTQ XL (ThermoFisher) interfaced with a 2D nanoLC system (See Eksigent website, www.eksigent.com). Peptides were fractionated by reversed-phase HPLC on a 75 um×100 mm C18 column (YMC ODS-AQ Sum particle size, 120 A pore size) with a 10 um emitter (New Objective) using 10-40% acetonitrile/0.1% formic acid gradient over 15 min at 300 nl/min. Using selected reaction monitoring (SRM), two transitions, 907.62 Da to 1110.5 (+/−1.0 Da) and 907.62 to 1181.5 (+/−1.0 Da), were monitored using source voltage 2.2 kV, collision energy 35, Q=0.250; activation time=30.

### Proliferation and Cytokine Production Assay

DR1 (DR B1*0101)-transgenic mice (Merck, West Grove, Pa.) expressing a fusion product of the DR1 binding groove and the membrane proximal domain of 1-E molecules were backcrossed to MHC class II KO mice for over 12 generations to eliminate endogenous class II proteins (I-A^{f}). The resulting mice express DR1 as their sole MHC class II molecule. DR1 transgenic mice were immunized with 50 μg native rHA1 or H5N1-rHA1 proteins in CFA in the base of the tail. After 8-10 days, the draining lymph nodes were harvested and the cells (˜4×10⁵) were incubated with a range of peptide and protein concentrations for 3 days before adding [³H]thymidine (Amersham). The cells were harvested and counted after a further incubation of 18-20 h, and the incorporated radioactivity was measured by Packard Matrix 96 beta counter. For each triplicate, lymphocyte proliferation was recorded as the mean counts per minute (cpm). For the cytokine assays, the draining lymph nodes of immunized mice were collected after 8-10 days and cells (5×10⁵-9×10⁵) were incubated with a range of peptide and proteins. Supernatants were collected after 24 h and 48 h incubation. IL-2 and IFN-γ concentrations were measured by enzyme-linked immunosorbent assay (ELISA) (R&D System). Optical density was measured with an ELISA reader (Dynex Technologies) with a test wavelength of 450 nm and a reference wavelength of 570 nm.

### Preparation of Tetramers and Tetramer Staining

Biotinylated DR1*0101/CLIP monomer was received from NIH tetramer core facility. The linker between CLIP peptide and DR113 chain was cleaved with thrombin for 2 h at room temperature in order to release the CLIP peptide. Peptide exchange was performed with thrombin cleaved complexes. Peptide exchange reactions were carried out with 3 uM thrombin cleaved DR/CLIP monomer and 50 uM HA₂₅₉₋₂₇₄ in a buffer containing 0.15M citrate phosphate pH5.5, 1% octylglucoside, 100 uM NaCl, 1 uM pepstatin (Sigma), 20 uM Leupeptin (Sigma), 500 uM Phenylmethanesulfonyl fluoride (Sigma), 2 mM EDTA (Sigma), and 50 uM AE206 helper peptide. The reactions were incubated for 3 days at 30° C. After this, the pH was adjusted to 7.5 and HA₂₅₉₋₂₇₄ loaded DR1 molecules were buffer-exchanged to PBS and concentrated using 10 kDa MWCO Microcon (Millipore). After concentration, peptide exchanged DR1 was tetramerized with PE labeled streptavidin (Invitrogen).

Cells were stained either with CLIP/DR1 tetramers or HA_{259-274/}DR1 tetramers for 2 h at 37° C. Then, they were stained with mAbs for CD4-FITC, CD8-eFlore 605, F4/80-APC, B220-APC, and CD44-Alexa 700 for additional 20 min at 4° C. 7-AAD was added 5 min before running the samples on flow cytometry. Cells were gated on lymphocytes by light scatter characteristics, then for CD4⁺ CD8⁻ CD4⁺ CD8⁻ F4/80⁻ B220⁻ 7AAD⁻ events. Numbers represent the percentage of tetramer-positive events among parent CD4 cells.

### Example 1

### Development of a Cell Free Antigen Processing System for the Detection of Immunodominant Epitopes

Antigen processing is complex and involves multiple steps, many chaperones, and several accessory proteins. For MHC class II processing, antigens are taken up by antigen presenting cells from exogenous sources and shuttled through a series of endosomal compartments. These compartments contain a denaturing environment, accessory chaperones, and proteolytic enzymes that digest protein antigens and allow binding of some peptide fragments to the groove of MHC class II molecules. To recreate the MHC class II antigen processing compartment, a minimum number of essential components were selected: a soluble form of the human MHC II molecule (HLA-DR1), soluble HLA-DR (DM), and cathepsins B, H, and S. DM was included in the assay because of its role in peptide editing. DM is known for catalyzing displacement of class II-associated invariant chain peptide (CLIP) and other peptides from the MHC groove as well as for inducing peptide binding. DM operates by generating a peptide-receptive MHC class II, which it accomplishes by exerting conformational changes in class II/peptide complexes through preventing the formation of a critical H-bond between βHis81 and the peptide main chain. By inducing MHC II to adopt a peptide-receptive conformation that can quickly sample a large pool of peptides derived from exogenously acquired proteins, DM acts as a peptide editor that, without being bound by theory, exerts influence on epitope selection.

Cathepsins D, L, and S are known to be involved in antigen processing. The role of cathepsin D is considered as dispensable in processing antigens, as APCs from cathepsin D deficient mice were not deficient in antigen presentation. Cathepsin L has a prominent role in antigen processing in the thymus. Cathepsin S is the major endoprotease involved in class II antigen processing outside of the thymus and can generate smaller fragments from full-length proteins, so was chosen as the only endoprotease. Exopeptidases, cathepsin B and cathepsin H, were chosen because they are constitutively expressed in all professional APCs and have carboxypeptidase (cathepsin B) and aminopeptidase (cathepsin H) activities important for trimming longer fragments bound to MHC II molecules. In addition, cathepsins B and H have endoprotease activity as well. In the antigen processing compartments, protein antigens are denatured and protein disulfide bonds are reduced by gamma-interferon-inducible lysosomal thiol reductase (GILT). To mimic the function of GILT, free L-cysteine was included in the assay to aid protein unfolding. L-cysteine also helps the catalytic activity of thiol-dependent lysosomal enzymes. Since cathepsins and DM are active in acidic pH, citrate phosphate buffer was used at pH 5.

The susceptibility of the soluble recombinant forms of HLA-DR1 (DR1) and DM to cathepsin B, H, and S were examined using SDS-PAGE assays.

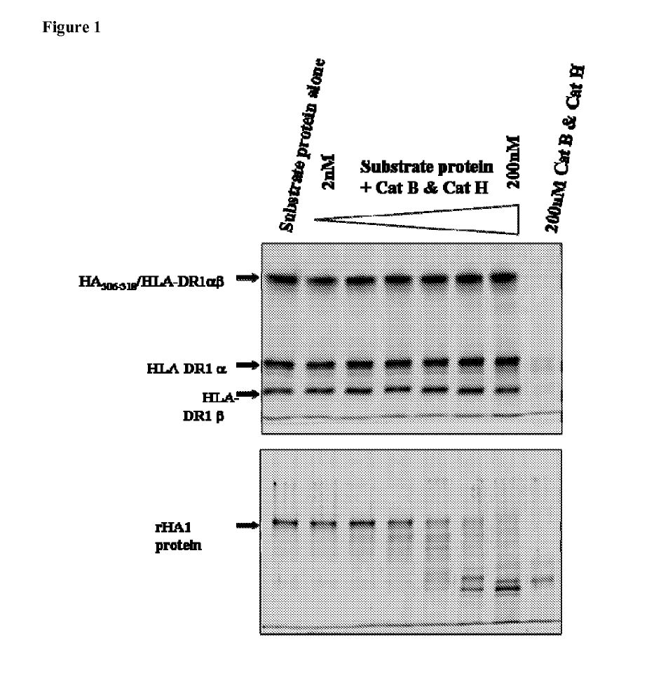

In the experiment depicted in FIG. 1, HA₃₀₆₋₃₁₈/DR1 complexes and recombinant influenza hemagglutinin were treated with cathepsins B (Cat B) and H (CatH) at various concentrations from 2 nM to 200 nM of each enzyme. The reactions were assembled in 0.15M citrate-phosphate buffer pH 5.2/10% sucrose/0.05% sodium azide/4 mM EDTA/6 mM L-Cysteine. After a 2 hour incubation at 37° C., samples were neutralized and resolved by SDS-PAGE under conditions that preserved the integrity of the trimeric peptide/DR1αβ complex.

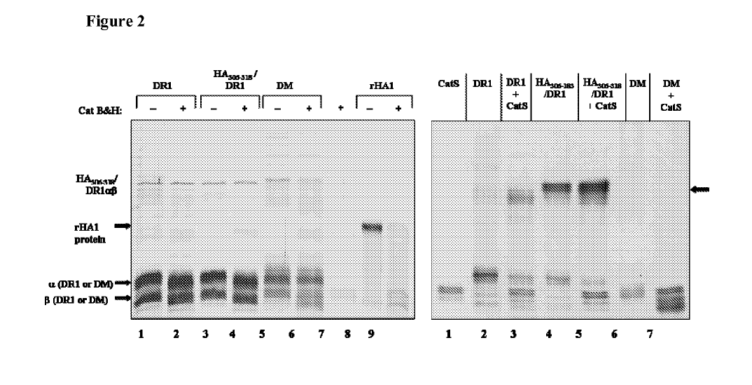

In the experiment depicted in the left panel of FIG. 2, empty DR1 molecules (lanes 1-2), pre-formed HA₃₀₆₋₃₁₈/DR1 complexes (lanes 3-4), and DM (lanes 5-6) were incubated in the presence or absence of 200 nM CatB and CatH for 2 h at 37° C. in the same buffer composition as in FIG. 1. After the incubation, samples were neutralized, mixed with standard Laemmli buffer, boiled and run on SDS-PAGE. rHA1 (lanes 8-9) served as a positive control for digestion by cathepsins B and H.

In the experiment depicted in the right panel of FIG. 2, empty DR1 (lane 3), HA₃₀₆₋₃₁₈/DR1 complexes (lane 5), and DM (lane 7) were treated with 100 nM cathepsin S (CatS). The reactions were assembled and run on gentle SDS-PAGE as in FIG. 1. All samples were resolved on a 12% acrylamide gel.

These SDS-PAGE assays showed little proteolysis of DR1 (either empty or peptide bound) to cathepsin B and H (FIGS. 1-2) under the conditions adopted for the digestion of HA1 recombinant protein (FIG. 1). Empty DR1 was susceptible to cathepsin S digestion, but peptide bound DR1 complex was resistant (FIG. 2). Soluble DM was somewhat sensitive to all three cathepsins. Because of cathepsin susceptibility of empty DR1 and DM, protein antigens were pre-incubated with DR1 and DM prior to the inclusion of the proteases.

To ensure efficient binding of peptides to soluble DR1 molecules, DR1 was induced to adopt a DR1_{rec} conformation in samples that did not contain DM. To achieve this, empty DR1 was pre-incubated with HA_{Y308A}, a variant of the HA₃₀₆₋₃₁₈ peptide that forms short-lived (t_{1/2}˜30 min) complexes with DR1 that upon dissociation induces a peptide-receptive conformation in MHC class II. Once reactions were set up containing all components of the system under endosomal/lysosomal-like conditions, peptides/DR1 complexes were isolated by immunoprecipitation and peptides eluted from DR1 and were analyzed on a Matrix-Assisted Laser Desorption Ionization (MALDI) LTQ ion trap mass spectrometer.

### Example 2

### Testing of the Cell Free Antigen Processing System Using HA1 of Influenza Hemagglutinin with a Known Immunodominant Epitope

The cell-free antigen processing system was tested using a protein with a well-defined, immunodominant epitope. This was done using a recombinant form of influenza hemagglutinin (rHA1) derived from strain A/PR/8/34, to which the A/Texas/1/77-derived HA₃₀₆₋₃₁₈ epitope was genetically attached near its C-terminus. This epitope, HA₃₀₆₋₃₁₈ (PKYVKQNTLKLAT), was initially found as immunodominant by testing T cell clones/lines generated from individuals infected with Influenza strain A/Texas/1/77 in response to synthesized overlapping peptides. HA₃₀₆₋₃₁₈ forms a stable complex with recombinant soluble DR1 (t_{1/2}˜6 days) and is resistant to DM-mediated dissociation.

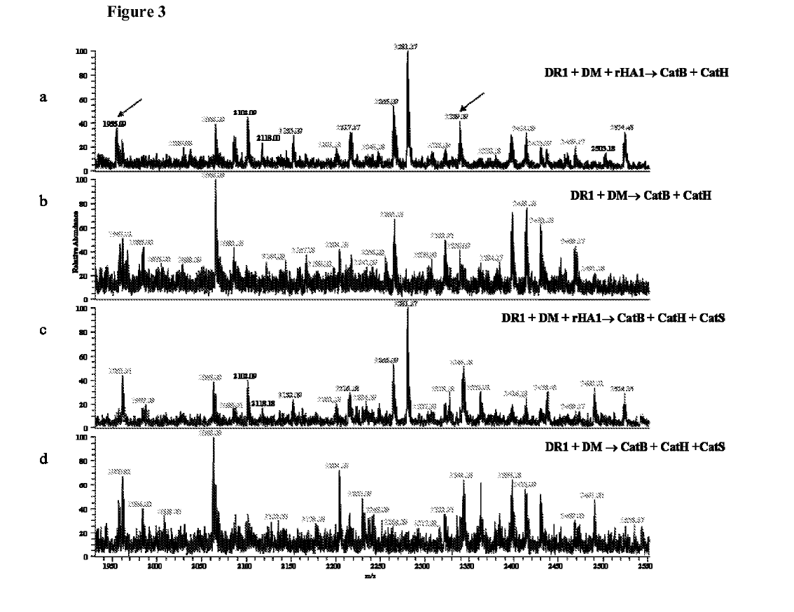

To test the system, rHA1 was incubated with DR1, DM, and cathepsins B and H, and peptides captured by DR1 were isolated and their identities were analyzed by mass spectrometry. Recombinant HA1 (rHA1), DR1, and DM were incubated together in citrate phosphate buffer pH 5 for 3 h at 37° C., and for an additional 2 h after the addition of CatB and CatH (FIG. 3 a-b), or CatB, CatH and CatS (FIG. 3. c-d). The molar ratio of components in the reactions were: 20 rHA1 :8.6 DR1:2.5 DM:2.3 CatB:2.3 CatH:1 CatS. Peptide/DR1 complexes were immunoprecipitated with Sepharose conjugated with anti-DR mAb (L243). Peptides were eluted from DR1 and analyzed by vMALDI.

The mass spectra of peptides eluted from DR1 after incubation with rHA1 in the presence of DM and cathepsins are shown in FIG. 3 a-d. Several rHA1-derived peptides were eluted from DR1 between m/z 1950-2530 Da (FIG. 3a) compared to the background spectrum (FIG. 3b). Sequences of 6 out of 10 peptide species were identified by collision induced dissociation (CID). Among those, 4 contained the immunodominant HA₃₀₆₋₃₁₈ epitope from Influenza strain A/Texas/1/77 (at 2153.09, 2217.27, 2281.27, 2524.45) (FIG. 3a). Sequences of the other two peptides at m/z 2265.09 and 2339.09 Da contained INSSLPYQNIHPVITIGECPK derived from influenza strain A/PR/8/34. For the last four peptides whose sequence identification posed a challenge (in black), a search was conducted to match their masses with sequences derived from HA1 protein using findpep tool (http://www.expasy.org/tools/findpept.html), while taking certain artifactual modifications into consideration. Peptides at m/z 1955.09, 2118.00, and 2503.18 Da each were predicted to contain peptides derived from influenza strain A/PR/8/34. FIG. 3c shows a second sample that included cathepsins B, H, and S that produced a profile nearly identical to that of the reaction containing cathepsins B and H only (FIG. 3a), with the exception that including cathepsins S caused elimination of rHA1 derived peptides at m/z 1955.09 Da and HA₂₉₈₋₃₁₇ (A/PR/8/34; INSSLPYQNIHPVITIGECPK) at m/z 2339.09 Da seen in FIG. 3a (shown by arrows).

To biologically verify the immunodominance of the identified peptides, DR1 (DR B1*0101) transgenic mice were used. These mice express a fusion product of DR1 groove and the membrane proximal domain of 1-E molecules. Transgenic mice were backcrossed to MHC class II KO mice for over 12 generations to eliminate endogenous class II proteins (1-A^{f}). Thus, these mice express DR1 as their only MHC class II.

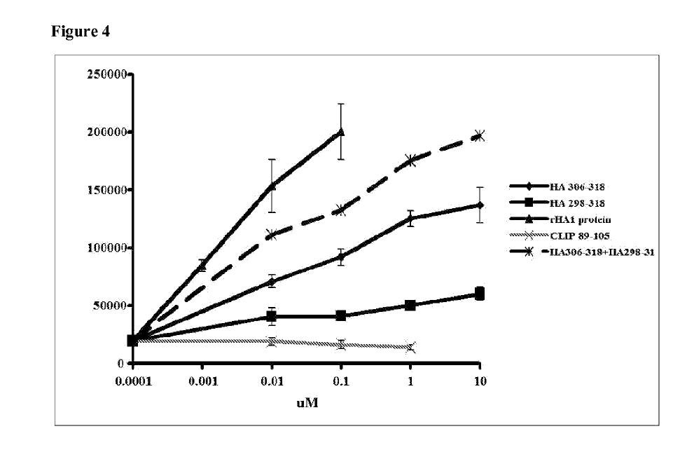

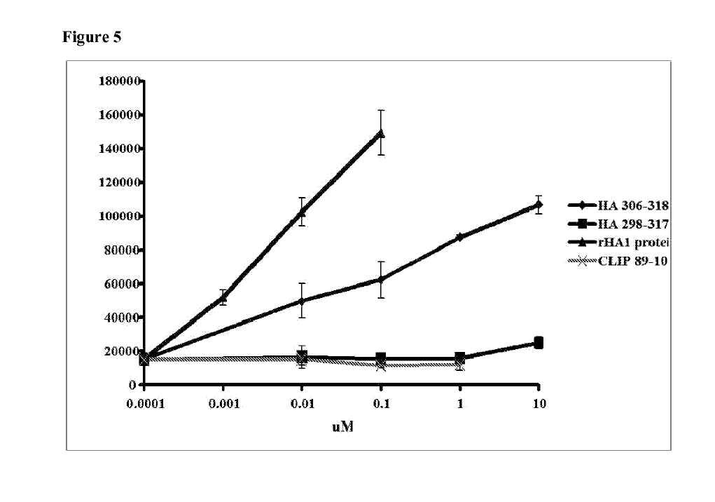

The DR1 transgenic mice were immunized with 50 μg of rHA1 protein in complete Freund's adjuvant (CFA) in the base of the tail. After 8-10 days, draining lymph nodes were harvested from immunized mice and the lymphoid cells were used in a recall proliferation assay using titrating doses of either the identified peptides or the rHA1 protein. Human short CLIP₈₉₋₁₀₅ (KMRMATPLLMQALPM) was used as a negative control. Strong dose dependent responses to HA₃₀₆₋₃₁₈ (A/Texas/1/77) and to the rHA1 protein not to human CLIP₈₉₋₁₀₅ were observed. There was a significantly lower (2 out of five mice) (FIG. 4) or no response (3 mice) to HA₂₉₈₋₃₁₇ (A/PR/8/34: INSSLPYQNIHPVITIGECPK) (FIG. 5). The sum of the proliferative responses to HA₃₀₆₋₃₁₈ and HA₂₉₈₋₃₁₇ peptides approached the magnitude of the response to the whole HA protein (FIG. 4, dashed line). Thus, the minimalist system could identify the immunodominant epitope of A/Texas/1/77, HA₃₀₆₋₃₁₈.

### Example 3

### Testing of the Cell Free Antigen Processing System Using Type II Collagen with a Known Immunodominant Epitope

The HA1 protein described above was a recombinant protein, with the known immunodominant epitope artificially attached to its end. Another protein that has a well-defined immunodominant epitope, was also tested type II collagen (CII). HLA-DR1 is a risk factor for the autoimmune disease rheumatoid arthritis (RA). CII, a major component of cartilage, is the main suspected autoantigen in RA induction. Through studies conducted on HLA-DR1 transgenic mice, the peptide containing residues 282 through 289 of CII (CII₂₈₂₋₂₈₉, FKGEQGPK), has been identified as its DR1-restricted immunodominant core epitope. In order to recapitulate physiological conditions of digesting this antigen, CII was pre-digested with matrix metalloproteinase 9 (MMP9) because it has been shown that CII undergoes extracellular processing first, and the resulting fragments are further processed in professional antigen presenting cells. Thus, CII pre-digested by MMP9 was included as a model antigen in the cell-free system along with other components.

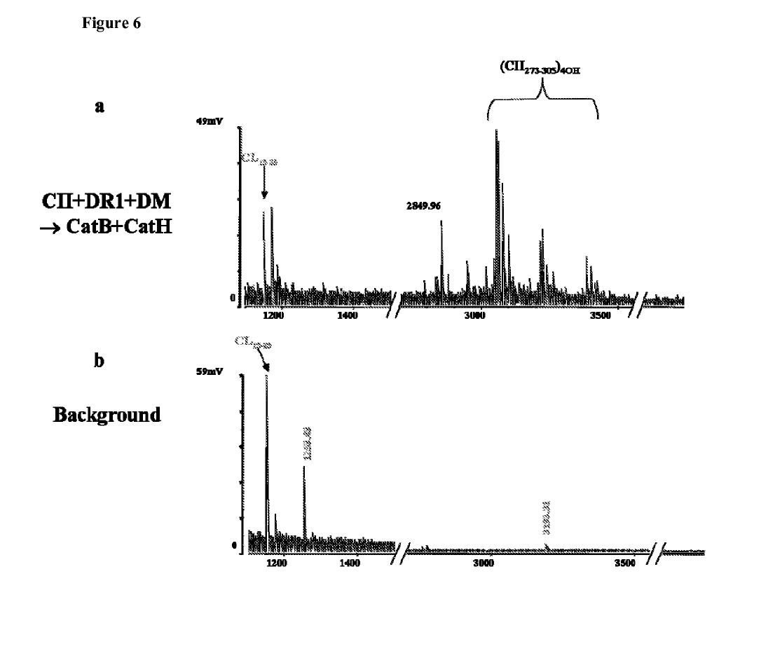

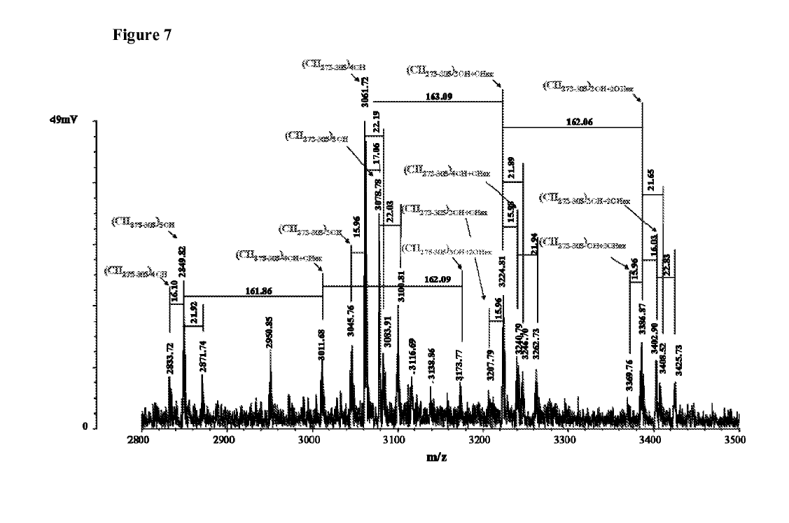

Peptide/DR1 complexes were then isolated by immunoprecipitation followed by peptide elution from DR1, and peptide identity was analyzed by mass spectrometry. The sample shown in FIG. 6a contained MMP9 digested CII in the reaction while the sample in FIG. 6b shows the background peaks since it did not include antigen in the reaction. The most noticeable difference between the two is that the majority of CII derived peptide species appeared on spectra between the m/z 3000-3500 Da range (FIG. 6-7). The most prominent peak of this cluster was sequenced by tandem mass spectrometry and determined to be residues 273-305 of CII (QTGEPGIAGFKGEQGPKGEPGPAGVQGAPGPAG) with four hydroxylated residues ((CII₂₇₃₋₃₀₅)_{4OH}). This fragment contains the core DR1-restricted immunodominant CII₂₈₂₋₂₈₉ epitope that is underlined. The other peptides in this cluster contained the same core epitope and were consistent in mass with post-translational modification (PTM) variants of this peptide that are expected to be present (FIG. 7).

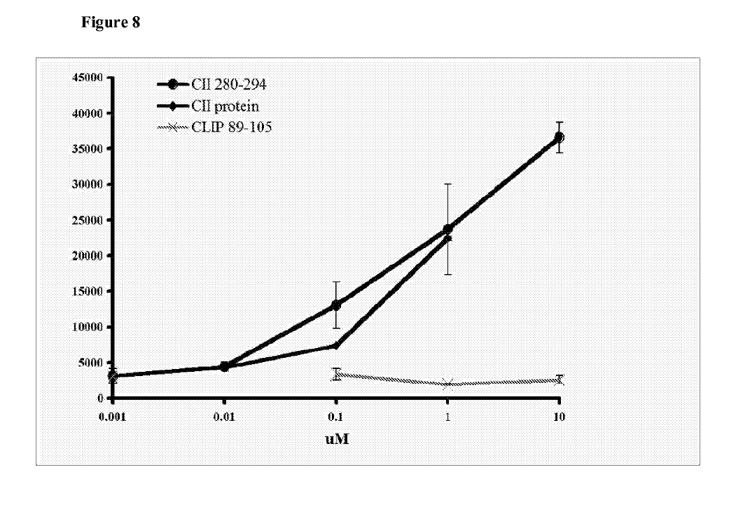

To evaluate the immunogenicity of the identified peptide from CII, DR1 transgenic mice were immunized with 50 ug of native bovine type II collagen in CFA in the base of the tail. After 8-10 days, lymph nodes were isolated from immunized mice and the lymphoid cells were used in a recall proliferation assay using titrating doses of synthesized peptide, CII₂₈₀₋₂₉₄ (AGFKGEQGPKGEPGP), the CII protein, or human CLIP₈₉₋₁₀₅. Strong dose dependent responses were observed to immunodominant epitope, CII₂₈₀₋₂₉₄ (AGFKGEQGPKGEPGP), and to the CII protein whereas no response to human CLIP₈₉₋₁₀₅ was measured (FIG. 8).

These data demonstrate that the cell-free antigen processing system selectively captures the physiological epitope from a protein, and that HLA-DR1 transgenic mice provide an in vivo model for confirmation of human T cell epitopes.

### Example 4

### De Novo Identification of Immunodominant CD4⁺ T Cell Epitopes from HA1 of the H5 Hemaggllutinin Protein

The immunodominant epitope of a novel antigen, HA1 protein of H5N1 influenza was identified. Highly pathogenic influenza A H5N1 viruses has caused disease outbreaks in poultry, wild birds, and humans in Asian countries with a fatality rate of approximately 60% and has been identified as a potential pandemic threat.

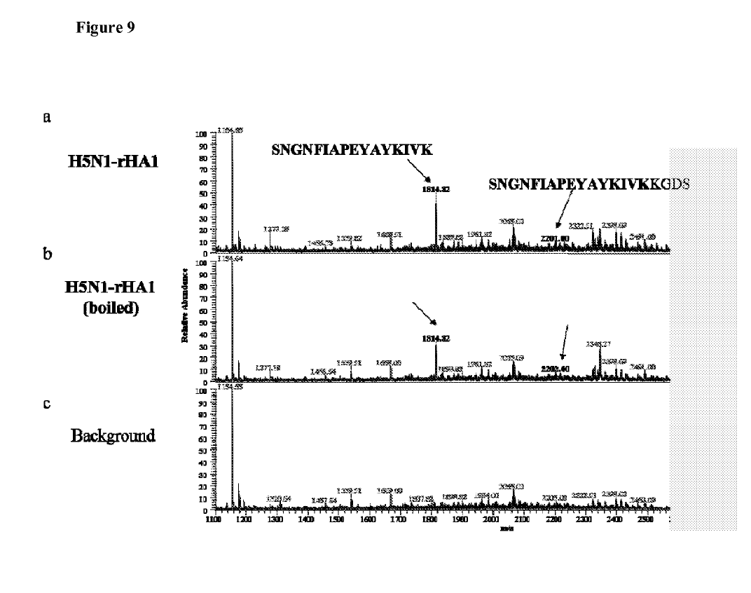

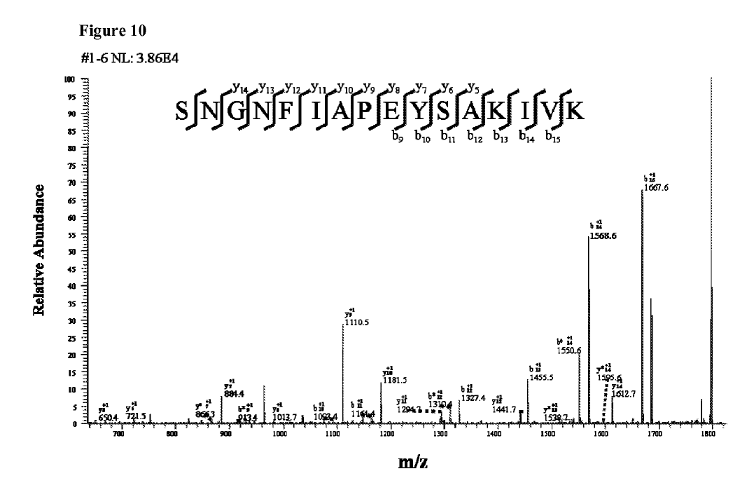

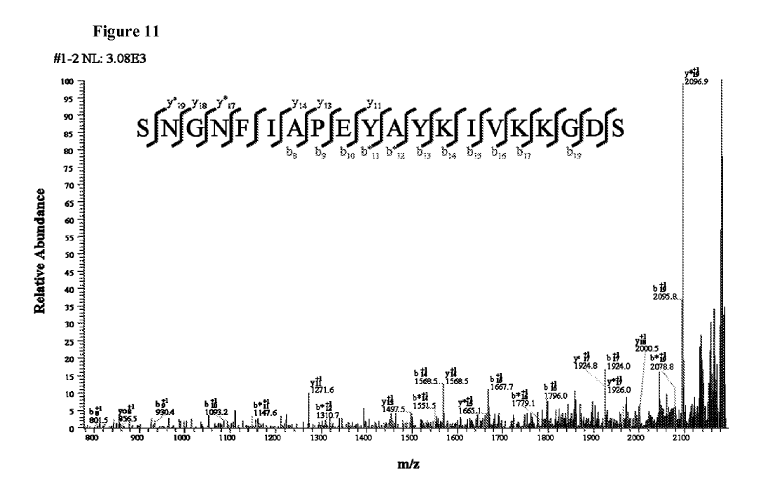

Either native or denatured HA1 protein from the H5N1 Influenza strain A/Vietnam/1203/2004 (H5N1 rHA1), which is highly conserved among H5 influenza strains, was incubated with DR1 and DM and then with cathepsins B, H, and S. Peptide/DR1 complexes were isolated and peptides were eluted from DR1. Peptide identities were then determined by mass spectrometry (FIG. 9-11). Two unique H5N1-rHA1 derived peptide species were detected at m/z˜1814.82 Da and 2201.00 Da (FIG. 9a-b) compared to background spectrum (FIG. 9c), regardless of whether the protein was native or heat denatured. Microsequencing of these peptides from CID spectra identified the sequences as HA₂₅₉₋₂₇₄ (SNGNFIAPEYAYKIVK) (FIG. 10), and HA₂₅₉₋₂₇₈ (SNGNFIAPEYAYKIVKKGDS) (FIG. 11) at m/z 1814.82 Da and m/z 2201.00 Da, respectively, both sharing the core 16-residue sequence (underlined). Thus, a single core epitope was selected from intact H5N1 rHA1.

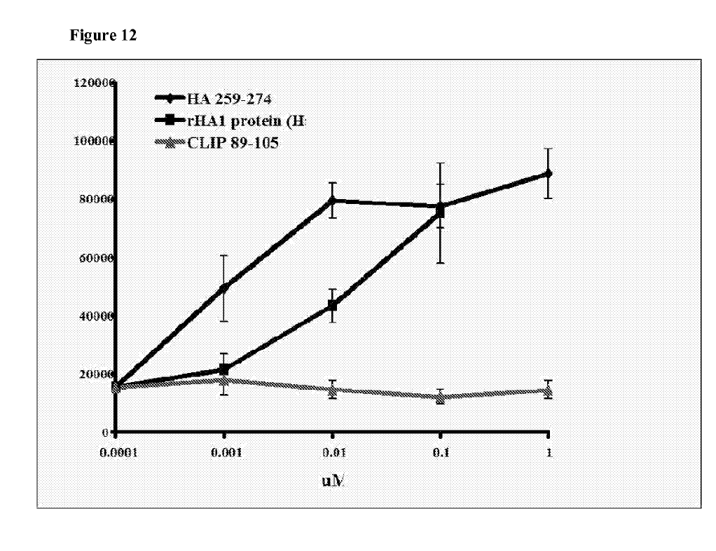

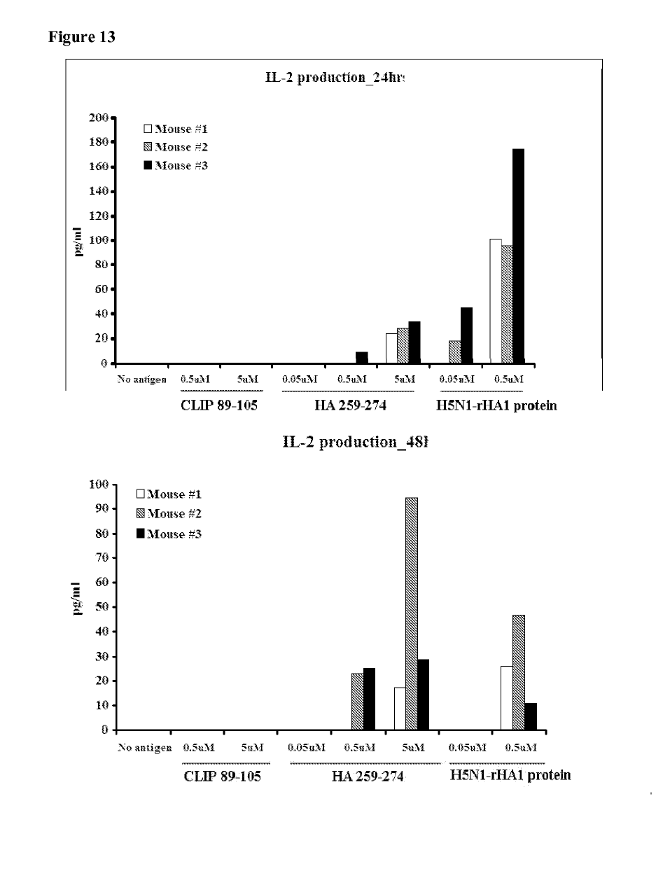

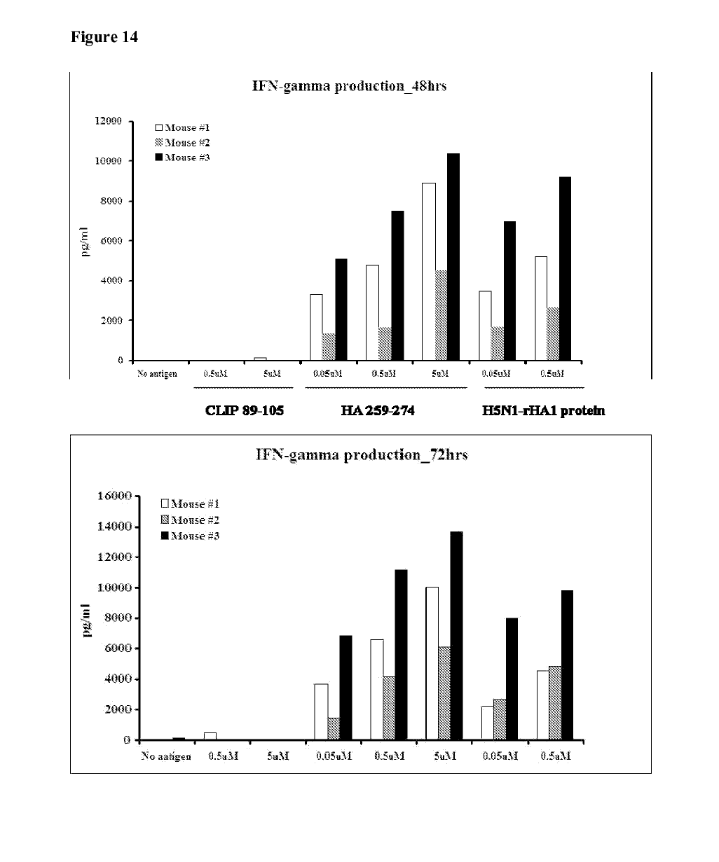

After sequence identification of the CD4⁺ T cell epitope using this cell-free system, the immunodominance of the identified peptide was confirmed by its ability to induce T cell responses. Six DR1 transgenic mice were immunized with 50 μg of H5N1-rHA1 protein in complete Freund's adjuvant at the base of the tail. After 8-10 days, draining lymph nodes were isolated and the lymphoid cells were used in proliferation and cytokine production assays using titrating doses of synthesized peptide, HA₂₅₉₋₂₇₄ (SNGNFIAPEYAYKIVK), human CLIP₈₉₋₁₀₅, or H5N1-rHA1 protein. Strong dose dependent proliferation responses to HA₂₅₉₋₂₇₄ and to the H5N1-rHA1 protein but not to CLIP₈₉₋₁₀₅ were observed (FIG. 12). Proliferation in response to HA₂₅₉₋₂₇₄ peptide was similar in magnitude to the response elicited by the whole H5N1-rHA1 protein. Cytokine production in supernatant of parallel culture wells taken after 24 hr and 48 hr in vitro culture for quantification of IL-2, or 48 hr and 72 hr for IFN-γ production, showed positive cytokine production in response to stimulation by the recombinant H5N1-rHA1 protein and HA₂₅₉₋₂₇₄ peptide, but not to stimulation by CLIP₈₉₋₁₀₅ (FIG. 13-14). IL-2 production in response to the H5N1-rHA1 was greater than to the HA₂₅₉₋₂₇₄ at 24 hr but its production reached similar level at 48 hr (FIG. 13). Overall, significantly higher amount of IFN-γ production was detected compared to the IL-2 by ELISA. IFN-γ production in response to stimulation by the H5N1-rHA1 protein and by the HA₂₅₉₋₂₇₄ peptide was similar in magnitude both at 48 hr and 72 hr (FIG. 14).

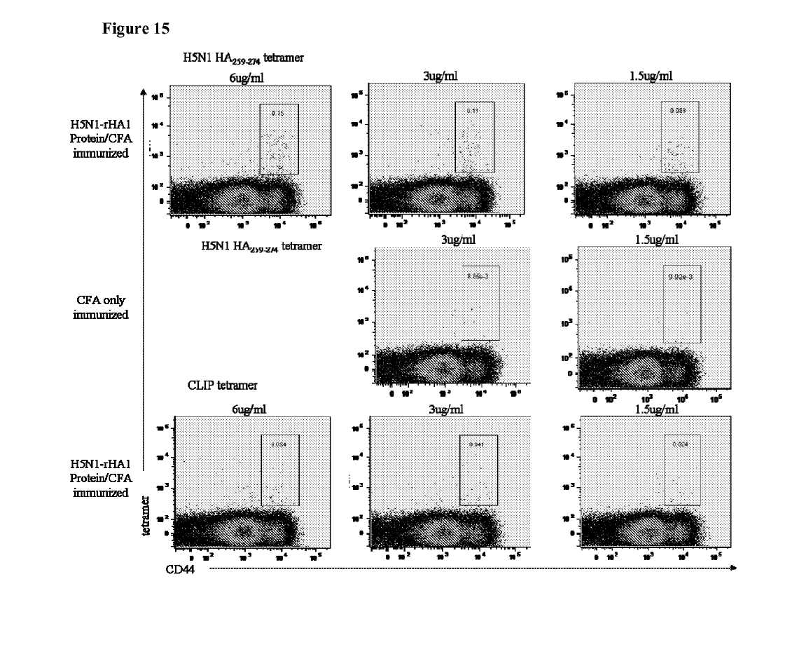

In order to further confirm the immunodominance of HA₂₅₉₋₂₇₄ peptide, DR1 transgenic mice were immunized either with the H5N1-rHA1 protein in the presence of CFA or with CFA alone. Draining lymph nodes freshly isolated from H5N1-rHA1 protein immunized mice (four mice pooled together) at day 8 were harvested and stained for HA₂₅₉₋₂₇₄/DR1 tetramers or CLIP/DR1 tetramers. Cells were stained for 2 h at 37° C. with varying concentrations of tetramer (12 ug/ml, 6 ug/ml, 3 ug/ml, or 1.5 ug/ml) followed by staining with mAbs for CD4-FITC, CD8-eFlore 605, CD44-Alexa 700, F4/80-APC, and B220-APC for additional 20 min at 4° C. (FIG. 15). The HA₂₅₉₋₂₇₄/DR1 tetramers positive population was ˜0.1% of the CD4⁺ CD44⁺ cells from mice immunized with H5N1-rHA1 protein/CFA compared to ˜0.01% HA₂₅₉₋₂₇₄/DR1 tetramer positive cells detected from CFA only immunized mice. An approximately 10-fold enrichment of HA₂₅₉₋₂₇₄ specific CD4 T cells from protein antigen immunized mice over cells from the CFA immunized mice was observed.

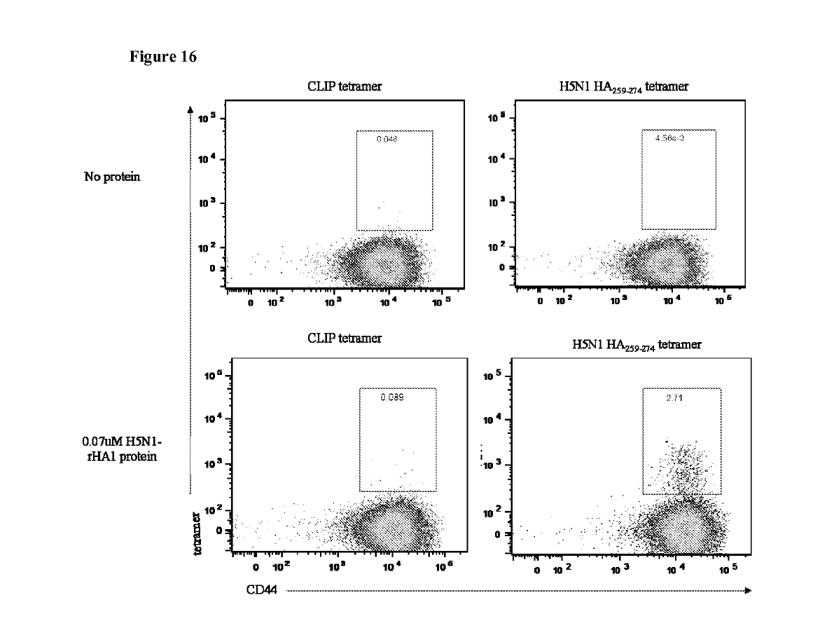

To expand the HA₂₅₉₋₂₇₄ specific CD4⁺ T cells, cells were stimulated with H5N1-rHA1 for additional 7 days and then stained either with HA₂₅₉₋₂₇₄/DR1 or CLIP tetramers (FIG. 16). After 7 days, additional stimulation with the protein, the HA₂₅₉₋₂₇₄/DR1 tetramers positive population was close to ˜3%. This shows about 30 fold increased staining of HA₂₅₉₋₂₇₄/DR1 tetramers compared to control CLIP/DR1 tetramers. An approximately 600-fold enrichment of HA₂₅₉₋₂₇₄ specific CD4⁺ T cells when cells were stimulated with protein compared to cells with media alone for 7 days in vitro.

These results from proliferation, cytokine measurements, and tetramer staining indicate that the cell-free antigen processing system identified an immunodominant epitope that activates CD4 T cells specific for HA1 from H5N1 influenza. Significantly, the identified epitope is present in most influenza strains that express H5 hemagglutinin, including H5N1, H5N2 and H5N3 influenza strains.

### EQUIVALENTS

Those skilled in the art will recognize, or be able to ascertain using no more than routine experimentation, many equivalents of the specific embodiments of the invention described herein. Such equivalents are intended to be encompassed by the following claims.

We claim:

1. An isolated polypeptide of less than or equal to 35 amino acids in length comprising an amino acid sequence that is at least 80% identical to a sequence selected from SEQ ID NOs: 1-29.

2. The polypeptide of claim 1, wherein said polypeptide is less than or equal to 30 amino acids in length.

3-5. (canceled)

6. The polypeptide of claim 1, wherein said amino acid sequence is at least 85% identical to a sequence selected from SEQ ID NOs: 1-29.

7-8. (canceled)

9. The polypeptide of claim 1, wherein said amino acid sequence is identical to a sequence selected from SEQ ID NOs: 1-29.

10-15. (canceled)

16. The polypeptide of claim 1, wherein said polypeptide is covalently bonded to a Class II MHC complex.

17. The polypeptide of claim 16, wherein said Class II MHC complex is HLA-DR.

18. The polypeptide of claim 17, wherein said HLA-DR is HLA-DR1.

19-24. (canceled)

25. A pharmaceutical composition comprising the polypeptide of claim 1 and a pharmaceutically acceptable carrier.

26-27. (canceled)

28. A method of raising an immune response in a subject comprising administering the pharmaceutical composition of claim 25 to the subject.

29-30. (canceled)

31. A method of reducing the risk of influenza infection in a subject comprising administering the pharmaceutical composition of claim 25 to the subject.

32-33. (canceled)

34. A method of treating a subject infected by influenza comprising administering the pharmaceutical composition of claim 25 to the subject.

35-38. (canceled)

39. A method of detecting exposure to influenza by a subject comprising the step of contacting CD4 T cells from the subject with the polypeptide covalently bonded to a Class II MHC of claim 16.

40-51. (canceled)

52. A kit comprising the pharmaceutical composition of claim 25.

53-54. (canceled)

55. An isolated nucleic acid encoding a polypeptide of claim 1, wherein said nucleic acid does not encode 36 consecutive amino acids of an HA1 of an influenza hemagglutinin.

56. The isolated nucleic acid of claim 55, wherein said polypeptide is at least 85% identical to a sequence selected from SEQ ID NOs: 1-29.

57-63. (canceled)

64. A vector comprising the nucleic acid of claim 55.

65. A pharmaceutical composition comprising the nucleic acid of claim 55.

66. A cell comprising the nucleic acid of claim 55.

67-69. (canceled)

70. The cell of claim 66, wherein said cell expresses HLA-DR.

71. (canceled)

72. A pharmaceutical composition comprising the cell of claim 70.


SEQUENCE LISTING (42 sequences)

SEQ ID NO: 1 - protein, 16 aa, Artificial Sequence
  Description of Artificial Sequence Synthetic peptide
SNGNFIAPEYAYKIVK

SEQ ID NO: 2 - protein, 20 aa, Artificial Sequence
  Description of Artificial Sequence Synthetic peptide
SNGNFIAPEYAYKIVKKGDS

SEQ ID NO: 3 - protein, 15 aa, Artificial Sequence
  Description of Artificial Sequence Synthetic peptide
SNGNFIAPEYAYKIV

SEQ ID NO: 4 - protein, 14 aa, Artificial Sequence
  Description of Artificial Sequence Synthetic peptide
SNGNFIAPEYAYKI

SEQ ID NO: 5 - protein, 13 aa, Artificial Sequence
  Description of Artificial Sequence Synthetic peptide
SNGNFIAPEYAYK

SEQ ID NO: 6 - protein, 15 aa, Artificial Sequence
  Description of Artificial Sequence Synthetic peptide
NGNFIAPEYAYKIVK

SEQ ID NO: 7 - protein, 14 aa, Artificial Sequence
  Description of Artificial Sequence Synthetic peptide
NGNFIAPEYAYKIV

SEQ ID NO: 8 - protein, 13 aa, Artificial Sequence
  Description of Artificial Sequence Synthetic peptide
NGNFIAPEYAYKI

SEQ ID NO: 9 - protein, 14 aa, Artificial Sequence
  Description of Artificial Sequence Synthetic peptide
GNFIAPEYAYKIVK

SEQ ID NO: 10 - protein, 13 aa, Artificial Sequence
  Description of Artificial Sequence Synthetic peptide
GNFIAPEYAYKIV

SEQ ID NO: 11 - protein, 13 aa, Artificial Sequence
  Description of Artificial Sequence Synthetic peptide
NFIAPEYAYKIVK

SEQ ID NO: 12 - protein, 19 aa, Artificial Sequence
  Description of Artificial Sequence Synthetic peptide
SNGNFIAPEYAYKIVKKGD

SEQ ID NO: 13 - protein, 18 aa, Artificial Sequence
  Description of Artificial Sequence Synthetic peptide
SNGNFIAPEYAYKIVKKG

SEQ ID NO: 14 - protein, 17 aa, Artificial Sequence
  Description of Artificial Sequence Synthetic peptide
SNGNFIAPEYAYKIVKK

SEQ ID NO: 15 - protein, 19 aa, Artificial Sequence
  Description of Artificial Sequence Synthetic peptide
NGNFIAPEYAYKIVKKGDS

SEQ ID NO: 16 - protein, 18 aa, Artificial Sequence
  Description of Artificial Sequence Synthetic peptide
GNFIAPEYAYKIVKKGDS

SEQ ID NO: 17 - protein, 17 aa, Artificial Sequence
  Description of Artificial Sequence Synthetic peptide
NFIAPEYAYKIVKKGDS

SEQ ID NO: 18 - protein, 15 aa, Artificial Sequence
  Description of Artificial Sequence Synthetic peptide
IAPEYAYKIVKKGDS

SEQ ID NO: 19 - protein, 14 aa, Artificial Sequence
  Description of Artificial Sequence Synthetic peptide
APEYAYKIVKKGDS

SEQ ID NO: 20 - protein, 13 aa, Artificial Sequence
  Description of Artificial Sequence Synthetic peptide
PEYAYKIVKKGDS

SEQ ID NO: 21 - protein, 18 aa, Artificial Sequence
  Description of Artificial Sequence Synthetic peptide
NGNFIAPEYAYKIVKKGD

SEQ ID NO: 22 - protein, 17 aa, Artificial Sequence
  Description of Artificial Sequence Synthetic peptide
NGNFIAPEYAYKIVKKG

SEQ ID NO: 23 - protein, 16 aa, Artificial Sequence
  Description of Artificial Sequence Synthetic peptide
NGNFIAPEYAYKIVKK

SEQ ID NO: 24 - protein, 17 aa, Artificial Sequence
  Description of Artificial Sequence Synthetic peptide
GNFIAPEYAYKIVKKGD

SEQ ID NO: 25 - protein, 16 aa, Artificial Sequence
  Description of Artificial Sequence Synthetic peptide
GNFIAPEYAYKIVKKG

SEQ ID NO: 26 - protein, 15 aa, Artificial Sequence
  Description of Artificial Sequence Synthetic peptide
GNFIAPEYAYKIVKK

SEQ ID NO: 27 - protein, 16 aa, Artificial Sequence
  Description of Artificial Sequence Synthetic peptide
NFIAPEYAYKIVKKGD

SEQ ID NO: 28 - protein, 15 aa, Artificial Sequence
  Description of Artificial Sequence Synthetic peptide
NFIAPEYAYKIVKKG

SEQ ID NO: 29 - protein, 14 aa, Artificial Sequence
  Description of Artificial Sequence Synthetic peptide
NFIAPEYAYKIVKK

SEQ ID NO: 30 - protein, 351 aa, Influenza A virus
MEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKHHHHHH

SEQ ID NO: 31 - protein, 13 aa, Artificial Sequence
  Description of Artificial Sequence Synthetic peptide
PKYVKQNTLKLAT

SEQ ID NO: 32 - protein, 13 aa, Artificial Sequence
  Description of Artificial Sequence Synthetic peptide
PKAVKQNTLKLAT

SEQ ID NO: 33 - protein, 15 aa, Artificial Sequence
  Description of Artificial Sequence Synthetic peptide
KMRMATPLLMQALPM

SEQ ID NO: 34 - protein, 15 aa, Artificial Sequence
  Description of Artificial Sequence Synthetic peptide
AGFKGEQGPKGEPGP

SEQ ID NO: 35 - protein, 21 aa, Artificial Sequence
  Description of Artificial Sequence Synthetic peptide
INSSLPYQNIHPVTIGECPKY

SEQ ID NO: 36 - protein, 6 aa, Artificial Sequence
  Description of Artificial Sequence Synthetic 6xHis tag
HHHHHH

SEQ ID NO: 37 - protein, 18 aa, Artificial Sequence
  Description of Artificial Sequence Synthetic peptide
MRGSHHHHHHTDPSSRSA

SEQ ID NO: 38 - protein, 27 aa, Artificial Sequence
  Description of Artificial Sequence Synthetic peptide
ACPKYVKQNTLKLATGMRKLHHHHHHN

SEQ ID NO: 39 - protein, 21 aa, Artificial Sequence
  Description of Artificial Sequence Synthetic peptide
INSSLPYQNIHPVITIGECPK

SEQ ID NO: 40 - protein, 8 aa, Artificial Sequence
  Description of Artificial Sequence Synthetic peptide
FKGEQGPK

SEQ ID NO: 41 - protein, 33 aa, Artificial Sequence
  Description of Artificial Sequence Synthetic polypeptide
QTGEPGIAGFKGEQGPKGEPGPAGVQGAPGPAG

SEQ ID NO: 42 - protein, 16 aa, Artificial Sequence
  Description of Artificial Sequence Synthetic peptide
SNGNFIAPEYSAKIVK